(19) United States
(12) Patent Application Publication
(10) Pub. No.: US 2018/0161032 A1
(43) Pub. Date: Jun. 14, 2018

(54) SUTURE FOR SOFT TISSUE FIXATION

(71) Applicant: Sinclair Pharmaceuticals Limited, Cheshire (GB)

(72) Inventor: Roberto Pizzamiglio, Marbella (ES)

(21) Appl. No.: 15/553,159

(22) PCT Filed: Feb. 23, 2016
(86) PCT No.: PCT/GB2016/050456

§ 371 Date: Aug. 23, 2017

(30) Foreign Application Priority Data
Feb. 27, 2015 (GB) 1503409.3

(51) Int. Cl. A61B 17/06

(52) U.S. Cl. CPC A61B 17/06166; A61B 2017/00004

(57) ABSTRACT

The present invention provides, in one aspect, a suture (100) for cosmetic surgery, aesthetic surgery or soft tissue fixation comprising a bioresorbable elongate filament structure (102) having first and second ends (104, 106); at least 10 bioresorbable elements (108) suitable for subcutaneous tissue engagement when in use, received movably upon the filament structure; wherein each of the elements is of a frusto- conical shape with a narrower end (202) and a broader end (204), having a hollow interior therethrough (206) enlarging outwardly toward the broader end, with the filament structure passing through said hollow interior; at least 12 protrusions (110) disposed along the filament structure; wherein each of the protrusions has a greater diameter than that of the hollow interior at the narrower end of each of the elements; and a first and a second needle (112, 114), coupled respectively to the first and second ends of the filament structure; wherein the elements and protrusions are disposed along the filament structure as spatially-separated first and second sets (116, 118), each of at least 5 elements and at least 6 protrusions; wherein the first set is proximal to the first needle and distal to the second needle, and wherein the second set is proximal to the second needle and distal to the first needle; wherein the elements are orientated in a bidirectional manner such that, when the filament structure is taut, the narrower ends of the elements in the first and second sets are orientated towards the first and second needles, respectively characterised in that, when the filament structure is taut, the length of filament structure between the narrower ends of the elements nearest to the first and second needles, when said elements are in the furthest position from their respective proximal needles that is allowed by the configuration of the suture (120, 122), and the points at which the filament structure is coupled to said needles (124, 126), is at least 10 cm (128, 130).

### FIELD OF THE INVENTION

The present invention relates to implantable, bioresorbable suture-based devices used for achieving the lift of subcutaneous, primarily facial, tissue.

### BACKGROUND TO THE INVENTION

The family of products sold under the trade name Silhouette Soft (as of February 2015) are fully bioresorbable, implantable suture-based devices, used for the lifting of facial tissue. They are disclosed in inter alia, U.S. Pat. No. 7,468,068 and U.S. Pat. No. 7,582,105 (Kolster).

Such devices are composed of single monofilament that is typically made up of 100% poly-L-lactic acid. Supported movably on each monofilament are two sets of 4 to 8 cones (elements which engage the subcutaneous tissue), depending on the configuration of the device, typically made from a copolymer of 82:18 (by mass ratio) L-lactide/ glycolide bioresorbable resin. The movement of the cones is restricted by a plurality of knots tied in the monofilament, of which there are two more than the number of cones. Furthermore, a 7 to 12 cm stainless steel suture needle is coupled to each end of the suture, via the method of crimping. The needle diameter is 23 Gauge.

As of February 2015, these devices are sold in three different formats, with 8, 12 or 16 cones. The 8 cone suture is approximately 30 cm in length, the 12 cone suture is approximately 27.5 cm in length, and the 16 cone suture is approximately 26.8 cm in length (not including the needles); although these values are subject to slight variance. During manufacture, each of the starting threads is pre-cut to the same length. The knots are then introduced into the monofilament manually. In the 8 cone variety, they are spaced approximately 5 mm apart, and separated spatially into two groups along the monofilament. In the 12 and 16 cone variety, they are similarly separated into groups, with spacings of approximately 8 mm.

During use, the insertion of the suture into the subcutaneous adipose tissue normally occurs through an entry point in the middle of the suture pattern. The central entry point enables the insertion of each half of the suture, with the direction of insertion of the two halves of the suture respecting the appropriate orientation of the cones. Different patterns (for example, linear or V-shaped) may be used depending on the area to be treated and the facial characteristics of the patient.

Such devices are an effective means of lifting subcutaneous tissue for a prolonged period of time.

### SUMMARY OF THE INVENTION

The present invention is based on the identification and solution of a novel problem, by the inventor, concerning the devices described above.

In some instances during the procedure, it has been found that the monofilament can detach from the needle before the exit of the needle from the skin is complete. This occurs at the point where the monofilament and needle are attached by crimping, and is thought to result from the resistance encountered when the cones pass through the entry point and begin to engage with the subcutaneous tissue of the patient. Accordingly, in such instances the end of the suture is lost under the skin, and the suture is no longer operable (i.e. it can no longer be brought into position). Therefore, the whole suture must likely be removed and the procedure repeated. This increases the time of the procedure and potentially the discomfort experienced by the patient. The problem has not been observed during use of the 8 cone variety of the devices described above.

The present inventor has realised that this problem may be due to the way the device is constructed. During manufacture as the number of cones increases, the amount of thread tied up in the knots increases, and the length of the final thread from end to end is reduced. The increase in overall length of the suture (from the 16 through to the 8 cone variety) is largely manifested as an increased length of filament between the points at which the ends of the filament are coupled to the needles and the knots closest to said needles. In the 8 cone variety, this length is approximately 12.0 cm; whereas in the 12 and 16 cone variety this is approximately 7.9 and 6.0 cm, respectively.

The above problem has been solved by the present inventor by increasing the length of the sutures having 10 or more cones. Specifically, an increase in the length of filament (to at least 10 cm) between the points at which the ends of the monofilament are coupled to the needles, and the cones nearest to said needles, when they are in the furthest position from said needles that is allowed by the configuration of the suture (when they are movably mounted on the filament), enables the leading ends of the suture to have already passed through the exit point (in the skin) before the cones begin to engage with the subcutaneous tissue. The cones assume the above position upon entry into the subcutaneous tissue, when their movement away from the needle is restricted by the protrusions positioned behind them (relative to the needle). This is when frictional forces between the cone and tissue increase, and detachment of the needle and monofilament at the crimping point can occur. However, with the invention, the leading ends of the suture will have already exited the skin before any potential detachment of the monofilament from the needle. The end of the suture can therefore be controlled by hand if such detachment were to take place, and it would not be necessary to repeat the procedure.

The present invention therefore mitigates the potential increased procedure time, cost and patient discomfort, which can result from occurrences of suture breakage as described above.

According to a first aspect of the invention, there is provided a suture (100) for cosmetic surgery, aesthetic surgery or soft tissue fixation comprising:
a bioresorbable elongate filament structure (102) having first and second ends (104, 106);
at least 10 bioresorbable elements (108) suitable for subcutaneous tissue engagement when in use, received upon the filament structure either movably or securely;
   wherein each of the elements is of a frusto-conical shape with a narrower end (202) and a broader end (204), having a hollow interior therethrough (206) enlarging outwardly toward the broader end, with the filament structure passing through said hollow interior;
at least 12 protrusions (110) disposed along the filament structure;
   wherein each of the protrusions has a greater diameter than that of the hollow interior at the narrower end of each of the elements; and
a first and a second needle (112, 114), coupled respectively to the first and second ends of the filament structure;

wherein the elements and protrusions are disposed along the filament structure as spatially-separated first and second sets (116, 118), each of at least 5 elements and at least 6 protrusions; wherein the first set is proximal to the first needle and distal to the second needle, and wherein the second set is proximal to the second needle and distal to the first needle;

wherein the elements are orientated in a bidirectional manner such that, when the filament structure is taut, the narrower ends of the elements in the first and second sets are orientated towards the first and second needles, respectively. characterised in that, when the filament structure is taut, the length of filament structure between the narrower ends of the elements nearest to the first and second needles, when said elements are in the furthest position from their respective proximal needles that is allowed by the configuration of the suture (120, 122), and the points at which the filament structure is coupled to said needles (124, 126), is at least 10 cm (128, 130).

According to a second aspect of the invention there is provided a suture (100) for cosmetic surgery, aesthetic surgery or soft tissue fixation comprising:
a bioresorbable elongate filament structure (102) having first and second ends (104, 106);
at least 10 bioresorbable elements (108) suitable for subcutaneous tissue engagement when in use, received upon the filament structure;
a first and a second needle (112, 114), coupled respectively to the first and second ends of the filament structure;

wherein the elements are disposed along the filament structure as spatially- separated first and second sets (116, 118), each of at least 5 elements; wherein the first set is proximal to the first needle and distal to the second needle, and wherein the second set is proximal to the second needle and distal to the first needle; characterised in that, when the filament structure is taut, the length of filament structure between the elements nearest to the first and second needles (120, 122), and the points at which the filament structure is coupled to said needles (124, 126), is at least 10 cm (128, 130).

### BRIEF DESCRIPTION OF THE FIGURES

FIG. 1 shows a suture (100) according to the present invention, having 12 bioresorbable elements (108) suitable for subcutaneous tissue engagement and 14 protrusions (110) in total. The first aspect of the invention requires said protrusions to be present, whereas these are not required by said second aspect. A bioresorbable elongate filament structure (102) is shown having first and second ends (104, 106), with the bioresorbable elements received thereon in a bidirectional manner, and the protrusions disposed along its length. The first aspect of the invention requires said elements to be movably received upon the filament structure; whereas according to the second aspect of the invention it is preferred that said elements are received securely. A first and a second needle are also shown (112, 114). Furthermore, two spatially-separated sets of 6 elements and 7 protrusions are indicated (116, 118). The characterising feature of the suture according to the invention is the length of filament structure (when said structure is taut) between the narrower ends of the elements nearest to the first and second needles, when said elements are in the furthest position from their respective proximal needles that is allowed by the configuration of the suture (120, 122), and the points at which the filament structure is coupled to said needles (124, 126); said length being at least 10 cm (124, 126).

FIG. 2 shows a single bioresorbable element (108) suitable for subcutaneous tissue engagement, indicating the narrower and broader ends (202, 204) of the frusto-conical shape of the element. The hollow interior of the element, which enlarges outwardly towards the broader end, is also indicated (206).

FIG. 3 shows a selection of 3 bioresorbable elements (108) and 3 protrusions, according to the specific embodiment of the invention where the protrusions are knots (302) in the filament structure (102).

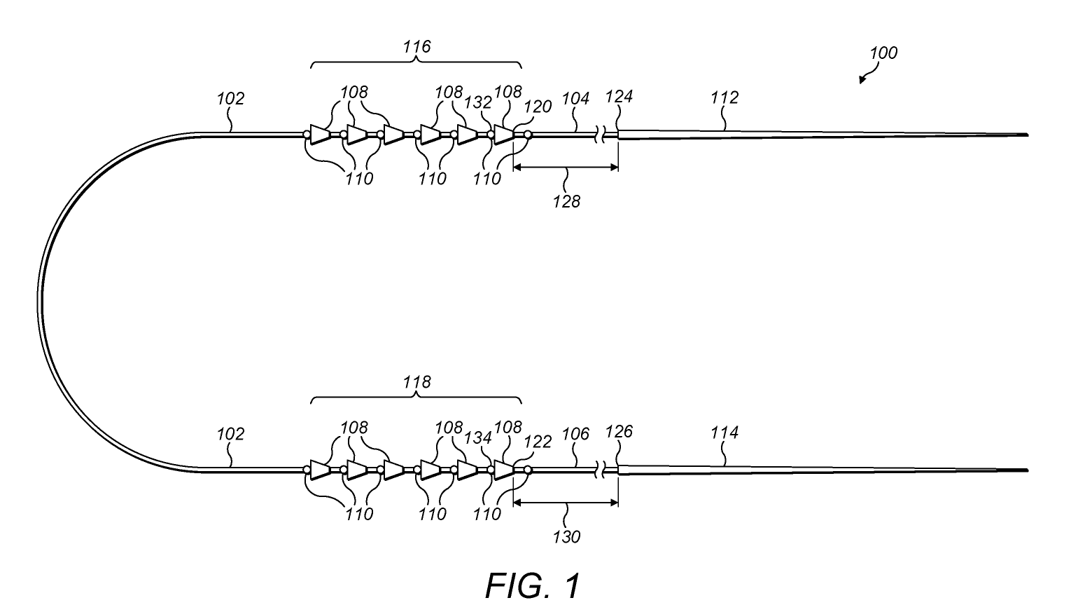

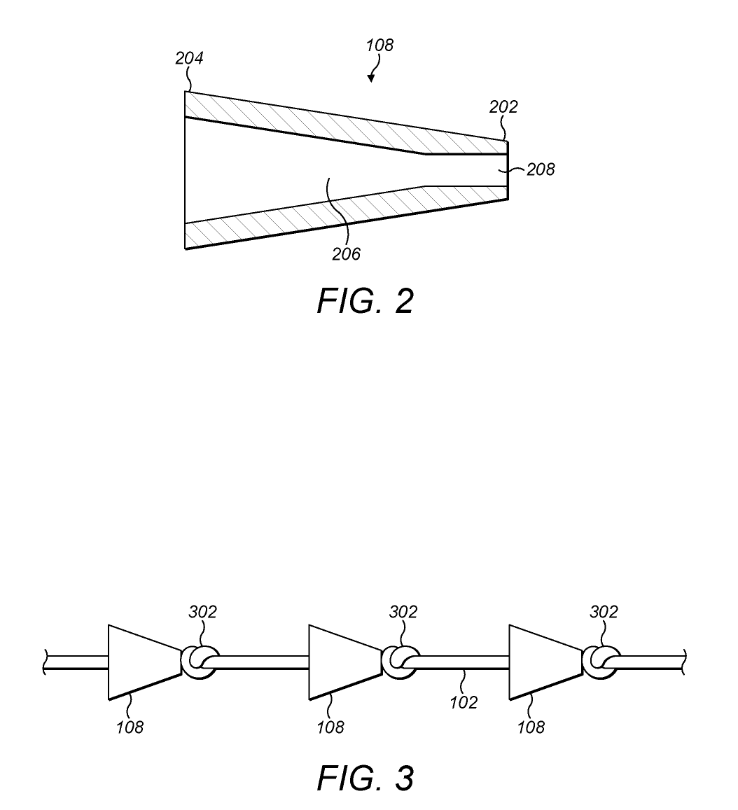

### DETAILED DESCRIPTION OF THE INVENTION

Devices sold under the trade name Silhouette Soft (as of February 2015) are described in inter alia, U.S. Pat. No. 7,468,068 and U.S. Pat. No. 7,582,105 (Kolster), which are herein incorporated by reference. Such devices are commercially available, to the skilled person, from Silhouette Lift, Inc. Furthermore, devices to be sold under the trade name Silhouette Instalift, which differ from Silhouette Soft only by the composition of the bioresorbable monofilament, are currently awaiting regulatory approval (as of February 2015).

According to said first and second aspects, the bioresorbable elongate filament structure (102) with first and second ends (104, 106) comprises any biocompatible material, preferably a polymer, capable of degradation and resorption when in situ. More preferably, the filament structure comprises, even more preferably consists of, either poly-L-lactic acid or a co-polymer of L-lactide and glycolide. Wherein said co-polymer is used, it is most preferred that L-lactide and glycolide are present at a mass ratio of 82:18, respectively. By way of example only, poly-L-lactic acid sold under the trade name Purasorb PL 32, and 82:18 L-lactide: glycolide resin sold under the trade name PURAC LG 8218 (both available from Corbion), may be used.

In one embodiment of said first and second aspects, the filament structure is a monofilament. In another embodiment of said first and second aspects, the filament structure is a multi-strand braided thread

Further according to said first and second aspects, received upon the filament structure are at least 10 bioresorbable elements (108), of a configuration suitable for engagement of the subcutaneous tissue of the patient when in situ. According to said first aspect, the bioresorbable elements are of a frusto-conical shape (as described in U.S. Pat. No. 7,582,105), thereby having a narrower and a broader end (202, 204), with a hollow interior passing longitudinally therethrough from said narrower to said broader end (206). According to said second aspect, the bioresorbable elements can be of any configuration suitable for subcutaneous tissue engagement when in situ, however it is preferred that they are of a frusto-conical shape, thereby having a narrower and a broader end (202, 204), with a hollow interior passing longitudinally therethrough from said narrower to said broader end (206). The hollow interior enlarges outwardly toward the broader end; and a portion of hollow interior beginning from the narrow end may define a cylindrical bore (208), prior to enlarging outwardly toward the broader end. The filament structure passes through said hollow interior, which is configured to accept it. According to said first aspect, the bioresorbable elements are movably mounted on the filament structure. According to said second aspect, it is especially preferred that the bioresorbable elements are securely mounted on the filament structure, such that when in use (that is, during the insertion procedure and after implantation in situ), the positions of the elements with respect to the filament structure are maintained. Further according to said first and second aspects, the bioresorbable elements comprise any biocompatible, preferably polymeric, material capable of degradation and resorption when in situ. More preferably, the bioresorbable elements comprise, even more preferably consist of, a co-polymer of L-lactide and glycolide; most preferably at a mass ratio of 82:18 L-lactide to glycolide. By way of example only, 82:18 L-lactide: glycolide resin sold under the trade name PURAC LG 8218 (available from Corbion) may be used.

In one embodiment of said first and second aspects there are at least 12 bioresorbable elements in total. In another embodiment of said first and second aspects, there are at least 16 bioresorbable elements. In two separate, but equally preferred embodiments of said first and second aspects, there are only 12 and only 16 bioresorbable elements.

Further according to said first aspect, disposed along the filament structure are at least 12 protrusions (110). The protrusions may be of a generally spherical nature. Alternatively, the protrusions may be knots (302), which are preferred when the filament is a polymeric monofilament. It is critical that the protrusions are of a larger diameter than that of the hollow interior at the narrower end of each of the bioresorbable elements, such that said narrower end of the element is unable to move past the protrusion. This restricts the movement of the elements. Therefore, they are able to engage and lift the subcutaneous tissue when implanted in situ.

In one embodiment of said first aspect, there are at least 14 protrusions in total. In another embodiment of said first aspect, there are at least 18 protrusions in total. In two separate, but equally preferred embodiments of first second aspect, there are only 14 and only 18 protrusions in total. In any given embodiment of said first aspect, it is especially preferred that the total number of protrusions is only two greater than the total number of bioresorbable elements on the suture. Further according to said first and second aspects, coupled to first and second ends of the filament structure are a first and a second needle (112, 114), respectively. Preferably, the needles comprise, more preferably consist of, stainless steel. A suitable diameter is 23 Gauge, and a suitable length is between 7 and 12 cm inclusive. The problem of breakage has been identified by the present inventor when using sutures where the ends of the filament structure have been coupled directly to the needles by crimping, a joining method known in the art.

Further according to said first aspect, the bioresorbable elements and protrusions are disposed along the filament structure as spatially-separated first and second sets (116, 118), of at least 5 elements and at least 6 protrusions. In one embodiment of said first aspect, there are at least 6 elements and at least 7 protrusions in each set. In another embodiment of said first aspect, there are at least 8 elements and at least 9 protrusions in each set. In two separate, but equally preferred embodiments of said first aspect, there are only 6 and only 8 elements, and only 7 and only 9 protrusions in each set for the two embodiments respectively (i.e. the sum of elements and protrusions in each set is 13 and 17 for each embodiment, respectively). The first set is proximal to the first needle and distal to the second needle, and the second set is proximal to the second needle and distal to the first needle. It is especially preferred that the protrusions are spaced in a serial arrangement within each set. In this regard, distances between protrusions in said serial arrangement of 0.4 cm to 1.0 cm are appropriate; for example, 0.8 cm. Furthermore, the elements are disposed in a bidirectional manner between the two sets. In this regard, as assessed when the filament structure is taut, the narrower ends of the bioresorbable elements in the first and second sets are orientated towards the first and second needles, respectively. With such a feature, the physician is able to redefine the area in which suture is implanted, by compressing the tissue around the centre point of the suture, causing the bioresorbable elements to engage with and lift the subcutaneous tissue.

Further according to said second aspect, the bioresorbable elements are disposed along the filament structure as spatially-separated first and second sets (116, 118), of at least 5 elements. In one embodiment of said second aspect, there are at least 6 elements in each set. In another embodiment of said second aspect, there are at least 8 elements in each set. In two separate, but equally preferred embodiments of said second aspect, there are only 6 and only 8 elements in each set for the two embodiments respectively. The first set is proximal to the first needle and distal to the second needle, and the second set is proximal to the second needle and distal to the first needle. It is especially preferred that the elements are disposed in a bidirectional manner between the two sets. In this regard, as assessed when the filament structure is taut, it is especially preferred that the narrower ends of the bioresorbable elements in the first and second sets are orientated towards the first and second needles, respectively. It is furthermore preferred that the bioresorbable elements are disposed in a serial arrangement within each set. In this regard, distances between elements in said serial arrangement of 0.4 cm to 1.0 cm are appropriate; for example, 0.8 cm.

Further according to said first aspect, the invention is characterised by a longer filament structure than existing 12 and 16 cone sutures. Specifically, the length of filament structure (measured when said structure is taut) between the narrower ends of the elements nearest to the first and second needles, when said elements are in the furthest position from their respective proximal needles that is allowed by the configuration of the suture (120, 122), and the points at which the filament structure is coupled to said needles (124, 126) is at least 10 cm (128, 130).

Preferably, this length is at least 11 cm. Alternatively, this length may be at least 12 cm, at least 13 cm, at least 14 cm, or at least 15 cm, depending on the nature of the procedure in which the invention is to be used. For the avoidance of doubt, the length of filament structure to be measured, when the filament structure is taut, is that which lies between the point, on the narrower end of the element that is nearest to the respective needle, that is both nearest to said needle and in contact with the filament structure (when the element is in the above position), and the point on said needle that is both in contact with the filament structure and nearest to said element.

Further according to said first aspect, the above distance is measured from the narrower ends of the elements nearest to the first and second needles, when the elements are in the furthest position from their respective proximal needles through coming into contact with the second nearest protrusions to said needles (132, 134). This will be the position of the elements when they first engage the subcutaneous tissue on entry, and furthermore, when frictional forces between the element and tissue will increase, potentially causing breakage at the point where the filament structure is coupled to the needle. Depending on the diameters of the protrusions, and the diameters of the hollow interiors of the bioresorbable elements at their broader ends, the second nearest protrusions to the first and second needles may, at least in part, be accepted within the hollow interior of the bioresorbable elements in question.

Further according to said second aspect, the invention is characterised by a longer filament structure than existing 12 and 16 cone sutures. Specifically, the length of filament structure (measured when said structure is taut) between the elements nearest to the first and second needles (120,122), and the points at which the filament structure is coupled to said needles (124, 126), is at least 10 cm (128, 130). Preferably, this length is at least 11 cm. Alternatively, this length may be at least 12 cm, at least 13 cm, at least 14 cm, or at least 15 cm, depending on the nature of the procedure in which the invention is to be used. For the avoidance of doubt, the length of filament structure to be measured, when the filament structure is taut, is that which lies between the point, on the element that is nearest to the respective needle, that is both nearest to said needle and in contact with the filament structure, and the point on said needle that is both in contact with the filament structure and nearest to said element. When the bioresorbable elements are securely received upon the filament structure (which is especially preferred according to said second aspect), the above distance is measured from the elements nearest to the first and second needles in their respective, non-variable, positions

The above feature of said first and second aspects mitigates the potential increased procedure time and patient discomfort, which can result from such occurrences of detachment.

1. A suture (100) for cosmetic surgery, aesthetic surgery or soft tissue fixation comprising:
a bioresorbable elongate filament structure (102) having first and second ends (104, 106);
at least 10 bioresorbable elements (108) suitable for subcutaneous tissue engagement when in use, received movably upon the filament structure;
wherein each of the elements is of a frusto-conical shape with a narrower end (202) and a broader end (204), having a hollow interior therethrough (206) enlarging outwardly toward the broader end, with the filament structure passing through said hollow interior;
at least 12 protrusions (110) disposed along the filament structure;
wherein each of the protrusions has a greater diameter than that of the hollow interior at the narrower end of each of the elements; and
a first and a second needle (112, 114), coupled respectively to the first and second ends of the filament structure;
wherein the elements and protrusions are disposed along the filament structure as spatially-separated first and second sets (116, 118), each of at least 5 elements and at least 6 protrusions; wherein the first set is proximal to the first needle and distal to the second needle, and wherein the second set is proximal to the second needle and distal to the first needle;
wherein the elements are orientated in a bidirectional manner such that, when the filament structure is taut, the narrower ends of the elements in the first and second sets are orientated towards the first and second needles, respectively characterised in that, when the filament structure is taut, the length of filament structure between the narrower ends of the elements nearest to the first and second needles, when said elements are in the furthest position from their respective proximal needles that is allowed by the configuration of the suture (120, 122), and the points at which the filament structure is coupled to said needles (124, 126), is at least 10 cm (128, 130).

2. A suture according to claim 1, wherein the filament structure has been coupled directly to the first and second needles by crimping.

3. A suture according to claim 1, wherein, when the filament structure is taut, the length of filament structure between the protrusions nearest to the first and second needles and the points at which the filament structure is coupled to said needles is at least 11 cm.

4. A suture according to claim 1, wherein the filament structure comprises a polymeric material.

5. A suture according to any preceding claim 1, wherein the filament structure comprises poly-L-lactic acid or a copolymer of L-lactide and glycolide.

6. A suture according to claim 1, wherein the bioresorbable elements comprise, preferably consist of, a copolymer of L-lactide and glycolide, more preferably at a mass ratio of 82:18 L-lactide: glycolide.

7. A suture according to claim 1, wherein the needles comprise, preferably consist of, stainless steel.

8. A suture according to claim 1, wherein the protrusions are spaced in a serial arrangement within each set (116, 118).

9. A suture according to claim 1, wherein the protrusions are knots (302) in the filament structure.

10. A suture according to claim 1, wherein the filament structure is a monofilament.

11. A suture according to claim 1, wherein the filament structure is a multi-strand braided thread.

12. A suture according to claim 1, comprising at least 12 of said elements and at least 14 of said protrusions.

13. A suture according to any preceding claim 1, comprising at least 16 of said elements and at least 18 of said protrusions.

14. A suture (100) for cosmetic surgery, aesthetic surgery or soft tissue fixation comprising:
a bioresorbable elongate filament structure (102) having first and second ends (104, 106);
at least 10 bioresorbable elements (108) suitable for subcutaneous tissue engagement when in use, received upon the filament structure;
a first and a second needle (112, 114), coupled respectively to the first and second ends of the filament structure;
wherein the elements are disposed along the filament structure as spatially-separated first and second sets (116, 118), each of at least 5 elements; wherein the first set is proximal to the first needle and distal to the second needle, and wherein the second set is proximal to the second needle and distal to the first needle;
characterised in that, when the filament structure is taut, the length of filament structure between the elements nearest to the first and second needles (120,122), and the points at which the filament structure is coupled to said needles (124, 126), is at least 10 cm (128, 130).

15. A suture according to claim 14, wherein the filament structure has been coupled directly to the first and second needles by crimping.

16. A suture according to claim 14 or 15, wherein each of the bioresorbable elements is of a frusto-conical shape having a narrower end (202) and a broader end (204); preferably wherein the elements are orientated in a bidirectional manner such that, when the filament structure is taut, the narrower ends of the elements in the first and second sets are orientated towards the first and second needles, respectively.

17. A suture according to claim 14, wherein the bioresorbable elements are received securely upon the filament structure such that, when in use, their positions with respect to the filament structure are maintained.
